(19) United States
(12) Patent Application Publication
(10) Pub. No.: US 2011/0230774 A1
(43) Pub. Date: Sep. 22, 2011

(54) DEVICE FOR MEASURING INFORMATION REGARDING BLOOD PRESSURE

(75) Inventors: Tatsuya Kobayashi, Otsu-shi (JP); Yoshihiko Sano, Kyoto-shi (JP); Hideaki Yoshida, Kyoto-shi (JP)

(73) Assignee: OMRON HEALTHCARE CO., LTD., Kyoto (JP)

(21) Appl. No.: 13/073,393

(22) Filed: Mar. 28, 2011

Related U.S. Application Data
(63) Continuation of application No. PCT/JP2009/065593, filed on Sep. 7, 2009.

(30) Foreign Application Priority Data
Sep. 26, 2008 (JP) 2008-248336

(51) Int. Cl. A61B 5/0225

(52) U.S. Cl. 600/493

(57) ABSTRACT

With a measurement device attached to the upper arm as a master, a measurement device, which is a slave, attached to the ankle is controlled to measure a pulse wave in synchronization. The measurement device on the master side obtains a measurement result from the measurement device on the slave side, and detects the appearance time difference of the pulse wave waveforms by synchronizing the pulse waves measured in both measurement devices, thereby calculating a Pulse Wave Velocity as the index of arterial sclerosis.

### TECHNICAL FIELD

The present invention relates to blood pressure information measurement devices and blood pressure information measurement systems, and in particular, to a blood pressure information measurement device and a blood pressure information measurement system for obtaining information regarding blood pressure and the degree of sclerosis of an artery from an index obtained by analyzing the pulse wave serving as the blood pressure information.

### BACKGROUND ART

Japanese Unexamined Patent Publication No. 2000-316821 (patent document 1) discloses a device for measuring the speed of propagation of the pulse wave ejected from the heart (hereinafter referred to as PWV: Pulse Wave Velocity) and determining the degree of arterial sclerosis as a device for measuring the degree of arterial sclerosis.

Japanese Unexamined Patent Publication No. 2002-143104 (patent document 2) discloses a device for obtaining the ratio between the upper arm blood pressure and the lower limb blood pressure.
Patent document 1: Japanese Unexamined Patent Publication No. 2000-316821
Patent document 2: Japanese Unexamined Patent Publication No. 2002-143104

The PWV is calculated from the appearance time difference of the respective pulse wave and the length of the artery between two points where the cuffs for measuring the pulse wave and the like are attached by attaching the cuffs on at least two or more areas such as the upper arm and the lower limb and measuring the pulse wave at the same time. Thus, the device of patent document 1 becomes large, the PWV is difficult to easily and conveniently measure at home because the cuffs and the like are to be attached to at least two areas, and the pulse wave is to be retrieved simultaneously from each cuff.

The device of patent document 2 also becomes large, the PWV is difficult to easily and conveniently measure at home because both cuffs are to be pressurized with one device, and the blood pressure of the upper arm and the blood pressure of the lower limb need to be simultaneously measured.

Therefore, one or more embodiments of the present invention provides a blood pressure information measurement device and a blood pressure information measurement system capable of using a plurality of blood pressure information measurement devices in one measurement to measure the blood pressure information with the respective device while synchronizing and accurately calculating the arterial sclerosis index with a simple configuration.

### SUMMARY OF INVENTION

According to one or more embodiments of the present invention, a blood pressure information measurement device includes a fluid bag, a measurement unit connected to the fluid bag for acquiring blood pressure information based on pressure change of the fluid bag, and a communication unit for communicating with another blood pressure information measurement device. The communication unit transmits a signal for instructing a start of measurement to the other blood pressure information measurement device, and acquires the blood pressure information measured by the other blood pressure information measurement device from the other blood pressure measurement device. The blood pressure information measurement device further includes a calculation unit for calculating an index of arterial sclerosis based on first blood pressure information, which is the blood pressure information measured by the measurement unit, and second blood pressure information, which is the blood pressure information measured by the other blood pressure information measurement device.

According to one or more embodiments of the present invention, the blood pressure information measurement device includes a fluid bag, a measurement unit for measuring a pulse wave based on a pressure change of the fluid bag, and a communication unit for communicating with another blood pressure information measurement device. The communication unit transmits a control signal for controlling an inner pressure of the fluid bag to the other blood pressure information measurement device. The blood pressure information measurement device further includes a calculation unit for calculating an index of arterial sclerosis from the pulse wave measured by the measurement unit while controlling the inner pressure of the fluid bag of the other blood pressure information measurement device with the control signal.

According to one or more embodiments of the present invention, a blood pressure information measurement system comprises a first blood pressure information measurement device and a second blood pressure information measurement device, wherein the first blood pressure information measurement device and the second blood pressure information measurement device acquire blood pressure information at different measurement sites of a same living body, and an index of arterial sclerosis of the living body is calculated based on the blood pressure information measured in the blood pressure information measurement devices in at least one blood pressure information measurement device of the first blood pressure information measurement device and the second blood pressure information measurement device.

According to one or more embodiments of the present invention, a plurality of areas can be compressed by the air bags to measure the blood pressure information while suppressing enlargement of the blood pressure information measurement device. An accurate index of arterial sclerosis thus can be obtained.

### BRIEF DESCRIPTION OF THE DRAWINGS

FIG. 1 is a view showing a specific example of an outer appearance of a blood pressure information measurement device (hereinafter referred to as measurement device) according to an embodiment.

FIG. 2 is a view showing a specific example of a correlation between an appearance time difference Tr between the ejection wave and the reflection wave, and the PWV.

FIG. 3 is a view showing the relationship of the measured pulse wave waveform, the ejection wave, and the reflection wave.

FIG. 4 is a block diagram showing function of the measurement device according to a first embodiment.

FIG. 5 is a view describing the measurement method using the measurement device according to the first embodiment.

FIG. 6 is a flowchart showing the measurement operation in the measurement device according to the first embodiment.

FIG. 7A is a view describing the measurement site in the measurement device according to the first embodiment.

FIG. 7B is a view describing the method of calculating the index of arterial sclerosis in the measurement device according to the first embodiment.

FIG. 8A is a view describing the measurement site in the measurement device according to the first embodiment.

FIG. 8B is a view describing the method of calculating the index of arterial sclerosis in the measurement device according to the first embodiment.

FIG. 9 is a block diagram showing function of the measurement device according to a second embodiment.

FIG. 10 is a view describing the measurement method using the measurement device according to the second embodiment.

FIG. 11 is a flowchart showing the difference of the measurement operation in the measurement device according to the second embodiment with the measurement operation in the measurement device according to the first embodiment.

FIG. 12 is a view showing a specific example of the measurement start signal and the synchronous pulse transmitted in step S85 during the measurement operation shown in FIG. 11.

FIG. 13A is a view describing the measurement result of the pulse wave in the measurement device according to the second embodiment.

FIG. 13B is a view describing the measurement result of the pulse wave in the measurement device according to the second embodiment.

FIG. 14 is a view describing the method of analyzing the pulse wave in the measurement device according to the second embodiment.

FIG. 15 is a view describing the measurement method using the measurement device according to a variant of the second embodiment.

FIG. 16 is a view showing function blocks of the measurement device according to a first variant.

FIG. 17 is a view showing a specific example of the relationship of the combination of measurement sites and the operation mode.

FIG. 18 is a flowchart showing the difference of the measurement operation in the measurement device according to a second variant with the measurement operation in the measurement device according to the first embodiment.

FIG. 19 is a view showing a specific example of a sphygmomanometer for ankle or wrist.

### DETAILED DESCRIPTION OF INVENTION

Embodiments of the present invention will be hereinafter described with reference to the drawings. The same reference numerals denote the same components and the configuring elements in the following description. The names and functions thereof are also the same.

The blood pressure information measurement device (hereinafter referred to as measurement device) 1, 2 according to one or more embodiments of the present invention will be described using FIG. 1. In the following description, the “blood pressure information” refers to information related to blood pressure that is obtained by measuring the living body. Specific examples of the “blood pressure information” include blood pressure value, pulse wave waveform, heart rate, and the like.

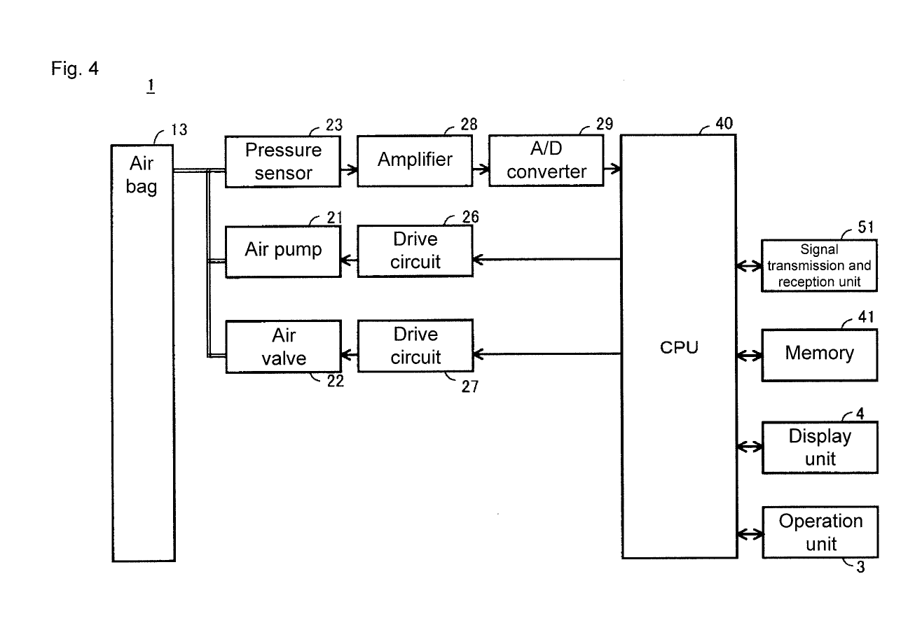

With reference to FIG. 1, a measurement device 1 according to a first embodiment or a measurement device 2 according to a second embodiment is connected with a cuff 9, to be attached to the measurement site, with an air tube 8. A display unit 4 for displaying various information including the measurement result and an operation unit 3 operated when giving various instructions to the measurement device 1, 2 are arranged on the front surface of the measurement device 1, 2. The operation unit 3 includes a switch 31, which is operated to turn ON/OFF the power supply, a switch 32, which is operated to instruct pressurization of an air bag 13 (FIG. 4) included in the cuff 9, a switch 33, which is operated to select whether the function of the measurement device 1, 2 is the master function or the slave function to be described later, and a switch 34, which is operated to select the measurement site to where the cuff 9 is attached. A connector 5 for connecting to other measurement devices is arranged on the side surface of the measurement device 1, 2. The information is exchanged with other measurement devices using a communication line connected to the connector 5. Wireless communication such as infrared communication may be carried out with the other measurement device instead of the wired communication. In this case, an infrared transmission and reception unit, and the like is arranged in place of the connector 5.

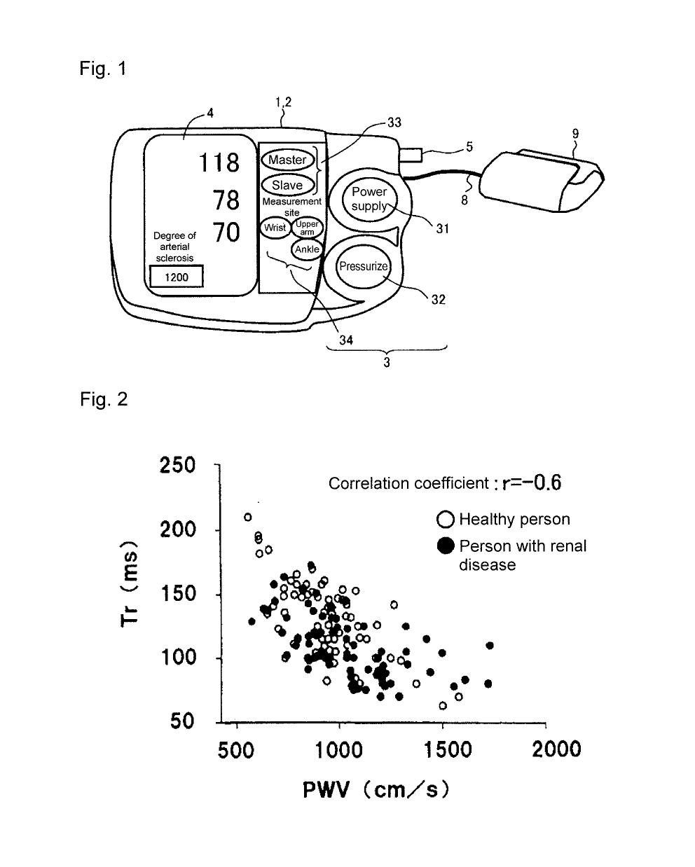

The measurement device 1, 2 obtains an index for determining the degree of arterial sclerosis based on the pulse wave waveform serving as the blood pressure information. As the speed of propagation of the pulse wave ejected from the heart (hereinafter referred to as PWV: Pulse Wave Velocity) becomes faster as the arterial sclerosis advances, the PWV acts as an index for determining the degree of arterial sclerosis. The appearance time difference Tr between the ejection wave and the reflection wave reflected and returned from the branch portion of the iliac artery is an index for determining the degree of arterial sclerosis using the PWV. The correlation between the appearance time difference Tr and the PWV is statistically obtained as shown in FIG. 2 if the individual parameters such as height and sex are obtained, as described in the document “Hypertension 1992 July; 20(1)” by London GM et al., (published on Jul. 20, 1992) P. 10 to 19. Therefore, the appearance time difference Tr between the ejection wave and the reflection wave can be an index for determining the degree of arterial sclerosis.

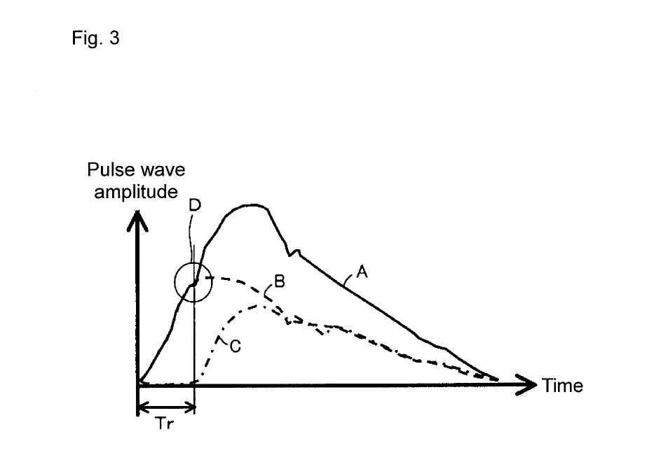

The principle for obtaining the index for determining the degree of arterial sclerosis based on the pulse wave waveform obtained from one measurement site will be described using FIG. 3. In FIG. 3, the waveform A shown with a solid line indicates the measured pulse wave waveform. The waveform B shown with a broken line indicates the ejection wave, and the waveform C shown with a chain dashed line indicates the reflection wave. As shown in FIG. 3, the pulse wave waveform A obtained by measurement is a synthetic wave of the ejection wave B and the reflection wave C. The arrival of the reflection wave at the measurement site is detected as an inflection point D in the pulse wave waveform A. Therefore, the appearance time difference Tr is obtained with the time from the rise of the pulse wave waveform A to the inflection point D. The accurate pulse wave waveform needs to be obtained to obtain the inflection point D from the pulse wave waveform A obtained by measurement. The accurate PWV can be obtained using the correlation relationship as shown in FIG. 2 by obtaining the accurate pulse wave waveform.

### First Embodiment

The function of the measurement device 1 will be described using FIG. 4. With reference to FIG. 4, the measurement device 1 includes an air pump 21, an air valve 22 and a pressure sensor 23 connected to the air bag 13 included in the cuff 9 through the air tube 8, as well as a CPU (Central Processing Unit) 40, a memory 41, and a signal transmission and reception unit 51. The memory 41 stores measurement results. Furthermore, the memory 41 stores a main program, a program for functioning as a master, and a program for functioning as a slave, to be described later, as a program to be executed in the CPU 40. The signal transmission and reception unit 51 is used to communicate with another measurement device using the communication line connected to the connector 5. The signal transmission and reception unit 51 transmits information input from the CPU 40 to another measurement device. The information received from another measurement device is output to the CPU 40.

The air pump 21 is driven by the drive circuit 26 that received the command from the CPU 40, and sends compressed gas to the air bag 13. The air pump 21 thereby pressurizes the air bag 13.

The open/close state of the air valve 22 is controlled by the drive circuit 27 that received the command from the CPU 40. The pressure in the air bag 13 is controlled when the open/close state of the air valve 22 is controlled. The air valve 22 thereby maintains or depressurizes the pressure of the air bag 13.

The pressure sensor 23 detects the pressure of the air bag 13. The pressure sensor 23 outputs a signal corresponding to a detection value to an amplifier 28. The amplifier 28 amplifies the signal input from the pressure sensor 23, and outputs to an A/D converter 29. The A/D converter 29 digitalizes the analog signal input from the amplifier 28, and outputs to the CPU 40.

The CPU 40 controls the drive circuits 26, 27 based on the command input to the operation unit 3. The CPU 40 also reads out the program stored in the memory 41 and executes the same to calculate the measurement value and the index, to be described later, using the value obtained from the pressure sensor 23 and/or the information received by the signal transmission and reception unit 51. The CPU 40 performs the process for displaying the calculation result on the display unit 4. The CPU 40 also performs the process for transmitting from the signal transmission and reception unit 51 to another measurement device. The process for storing in a predetermined region of the memory 41 is also performed.

The drive circuits 26, 27, the amplifier 28, the A/D converter 29, the memory 41, and the signal transmission and reception unit 51 all may be functions realized with the hardware configuration different from the CPU 40, or at least one may be a function exhibited by the CPU 40 when the CPU 40 executes the program.

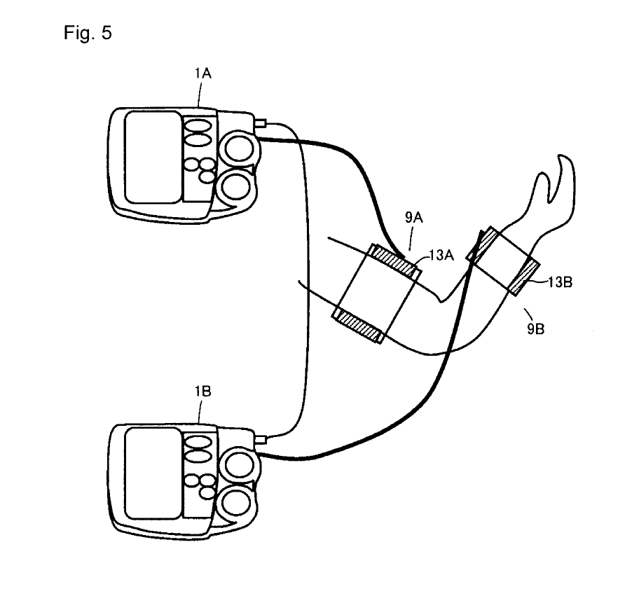

The measurement method using the measurement device 1 will now be described using FIG. 5. With reference to FIG. 5, two connected measurement devices 1 represented as the measurement devices 1A, 1B are used in the first embodiment, and are operated in cooperation with each other to obtain the blood pressure information and calculate the index of arterial sclerosis. In the case shown in FIG. 5, the measurement device 1A functions as a master, and the measurement device 1B functions as a slave. The measurement device 1A, which is a master, has the cuff 9A to be attached to the upper arm on the central side, and the measurement device 1B, which is the slave, has the cuff 9B to be attached to the peripheral side than the cuff 9A of the same arm. In the example of FIG. 5, the cuff is attached to the wrist, but the cuff 9B may be attached to any site as long as it is on the peripheral side than the cuff 9A of the same arm, as will be described later using the figures.

The cuff 9 internally includes the air bag 13 serving as a fluid bag for compressing the living body and measuring the blood pressure and the pulse wave serving as the blood pressure information. The air bag 13A included in the cuff 9A compresses the central side, and the air bag 13B included in the cuff 9B compresses the peripheral side. The measurement device 1A that functions as the master also functions as a control device for controlling the measurement device 1B that functions as a slave. The measurement device 1A that functions as a master also calculates the measurement value and the index using the own measurement result and the measurement result in the measurement device 1B that functions as the slave, and outputs the calculation result.

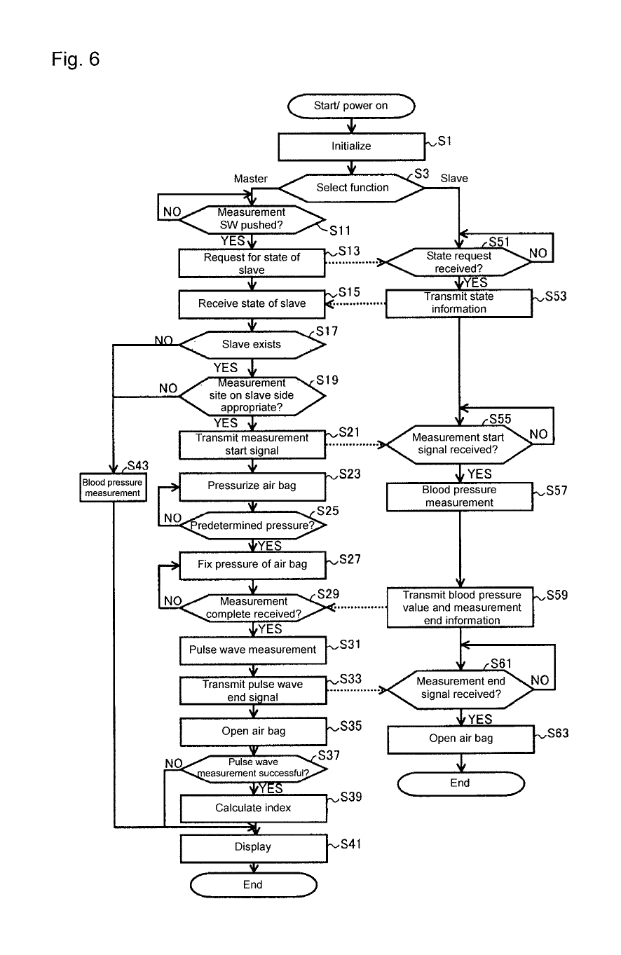

The measurement operation in the measurement device 1 will be described using FIG. 6. The operation shown in FIG. 6 starts when the switch 31 is pushed to turn ON the power arranged on the operation unit 3, and is realized when the CPU 40 reads out the program stored in the memory 41 and controls each unit shown in FIG. 2.

With reference to FIG. 6, when the operation starts, the CPU 40 reads out the main program from the memory 41 and executes the same, and initializes each unit in step S1. In step S3, the CPU 40 determines which function, the master function or the slave function, is selected based on the operation signal from the switch 33, and reads out the program corresponding to the selected function from the memory 41 and executes the same. In other words, if it is determined that the master function is selected with the switch 33 (“master” in step S3), the CPU 40 reads out the program for causing the measurement device 1 to function as the master from the memory 41, and executes the same. The measurement device 1 thereafter performs the operation of the measurement device 1A on the master side. If it is determined that the slave function is selected (“slave” in step S3), the CPU 40 reads out the program for causing the measurement device 1 to function as the slave from the memory 41, and executes the same. The measurement device 1 thereafter performs the operation of the measurement device 1B on the slave side. Thus, the aspect that the measurement device operates as the measurement device on the master side or the measurement device on the slave side by reading the program corresponding to the selected function and branching the subsequent operation is the same in the second embodiment and the variant, to be described later.

If the measurement device 1 functions as the master, that is, if the measurement device 1 is the measurement device 1A on the master side in the example of FIG. 5, the CPU 40 monitors the input of the operation signal from the switch 32 for pressurizing the air bag 13A of the cuff 9 and starting the measurement, and waits until the switch 32 is pushed. When it is determined that the switch 32 is pushed (YES in step S11), the CPU 40 transmits predetermined information for requesting the state to the other measurement device 1 connected with the connector 5 from the signal transmission and reception unit 51 in step S13.

If the measurement device 1 functions as the slave, that is, if the measurement device 1 is the measurement device 1B on the slave side in the example of FIG. 5, the CPU 40 waits until receiving the request transmitted in step S13 from the measurement device 1A on the master side with the signal transmission and reception unit 51. When receiving the request with the signal transmission and reception unit 51 (YES in step S51), the CPU 40 transmits information for notifying the state of the measurement device 1B to the measurement device 1A connected with the connector 5 from the signal transmission and reception unit 51 in step S53. The information transmitted here at least includes information indicating the measurement site selected with the switch 34 in the measurement device 1B.

In the measurement device 1A on the master side, when the signal transmission and reception unit 51 receives the information transmitted from the measurement device 1B in step S53 in step S15, the content of the relevant information is analyzed in the CPU 40. Specifically, whether the measurement device 1B functioning as the slave exists, and the measurement site on the slave side is appropriate are determined in the CPU 40. Whether or not the measurement device 1B exists may be determined by receiving the information transmitted in step S53, or by a signal contained in the information indicating that the relevant measurement device (measurement device 1B) functions as the slave. Furthermore, as the relevant information contains the information indicating the measurement site selected with the measurement device (measurement device 1A), determination may be made that the other measurement device 1 is the measurement device 1B that functions as the slave from the relationship with the measurement site selected with the measurement device (measurement device 1A). That is, when the measurement site selected with the other measurement device 1 is on the peripheral side than the measurement site selected with the measurement device 1A, the CPU 40 can determine that the other measurement device 1 is the measurement device 1B that functions as the slave. Alternatively, the CPU 40 may store the measurement site to be selected with the measurement device 1B that functions as the slave in advance, and determine that the other measurement device 1 is the measurement device 1B that functions as the slave when the information indicating the measurement site contained in the information represents the stored measurement site.

In the measurement device 1A on the master side, when it is determined that the measurement device 1B that functions as the slave exists and the measurement site on the slave side is appropriate (YES in step S17 and YES in step S19) by the CPU 40, the CPU 40 outputs a signal instructing the start of the blood pressure measurement from the signal transmission and reception unit 51 to the measurement device 1B on the slave side in step S21.

In the measurement device 1A on the master side, when it is determined that the measurement device 1B that functions as the slave does not exist (NO in step S17) by the CPU 40, the relevant measurement device functions as a normal blood pressure measurement device. In other words, the CPU 40 performs the blood pressure measurement operation in step S43, and performs the process for displaying the measurement result on the display unit 4 in step S41, and terminates the process. When it is determined that the measurement site is not appropriate even if the measurement device 1B on the slave side exists (YES in step S17 and NO in step S19), the relevant measurement device similarly functions as the normal blood pressure measurement device, and the CPU 40 performs the blood pressure measurement operation in step S43 and performs the process for displaying the measurement result on the display unit 4 in step S41 and terminates the process.

In the measurement device 1B on the slave side, when the signal instructing the start of measurement transmitted from the measurement device 1A on the master side is received by the signal transmission and reception unit 51 in step S21 (YES in step S55), the CPU 40 starts the blood pressure measurement operation in step S57. In this case, the measurement device 1B on the slave side notifies the start of the blood measurement operation to the measurement device 1A on the master side.

In the measurement device 1A on the master side, when the blood pressure measurement operation in the measurement device 1B on the slave side starts in step S57, the CPU 40 outputs a control signal to the drive circuit 26A to start the pressurization of the air bag 13A included in the cuff 9A in step S23. The pressurization of the air bag 13A in step S23 is carried out until it is determined by the CPU 40 that the pressure of the air bag 13A obtained from the pressure sensor 23A reached a predetermined pressure. When the pressure of the air bag 13A reaches a predetermined pressure (YES in step S25), the CPU 40 fixes the inner pressure of the air bag 13A to the predetermined pressure in step S27.

The measurement method carried out in the normal sphygmomanometer is adopted for the measurement of the blood pressure in the measurement device 1B on the slave side in step S57. Specifically, the CPU 40 outputs the control signal to the drive circuit 26A and gradually pressurizes the inner pressure of the air bag 13B. The CPU 40 calculates the diastolic blood pressure value and the systolic blood pressure value based on the pressure signal obtained from the pressurization sensor 23A in the pressurization process. After the measurement of the blood pressure in step S57 is completed, the CPU 40 transmits the information including the calculated blood pressure value and the signal indicating that the measurement completed to the measurement device 1A on the master side from the signal transmission and reception unit 51 in step S59.

In the measurement device 1A on the master side, the inner pressure of the air bag 13A is fixed at the predetermined pressure until receiving the information transmitted from the measurement device 1B on the slave side in step S59. When the signal transmission and reception unit 51 receives the information (YES in step S29), the CPU 40 measures the pulse wave in step S31. Meanwhile, the inner pressure of the air bag 13B is maintained at the inner pressure at the time point the blood pressure measurement in step S57 is terminated in the measurement device 1B on the slave side. That is, the pulse wave is measured in the measurement device 1A on the master side with the cuff 9B on the slave side applied to the attachment site.

In the measurement device 1A on the master side, after the pulse wave measurement in step S31 is finished, the CPU 40 notifies the end of the pulse wave measurement to the measurement device 1B on the slave side with the signal transmission and reception unit 51 in step S33. Thereafter, the CPU 40 outputs a control signal to the drive circuit 27A to open the air bag 13A in step S35.

When the pulse wave is measured in step S31 and the measurement is finished (YES in step S37), the CPU 40 calculates the index of arterial sclerosis from the measurement result and the attachment site of the cuff 9 in step S39. The specific content in step S39 will be described later. In step S41, the CPU 40 performs the process for displaying the blood pressure received from the measurement device 1B on the slave side in step S29, the measurement result of the pulse wave in step S31, and the index calculated in step S39 on the display unit 4 to display the same, and terminates the series of processes.

If the measurement is terminated without the pulse wave measurement in step S31 (NO in step S37), the CPU 40 does not perform the process for calculating the index in step S39, and performs the process for displaying a warning that the pulse wave was not measured on the display unit 4 in step S41 and terminates the series of processes. In this case, the blood pressure value received from the measurement device 1B on the slave side in step S29 may be displayed.

In the measurement device 1B on the slave side, when receiving the notification that the pulse wave measurement is completed from the measurement device 1A on the master side in step S33 (YES in step S61), the air bag 13B is similarly opened in step S63 and the process is terminated.

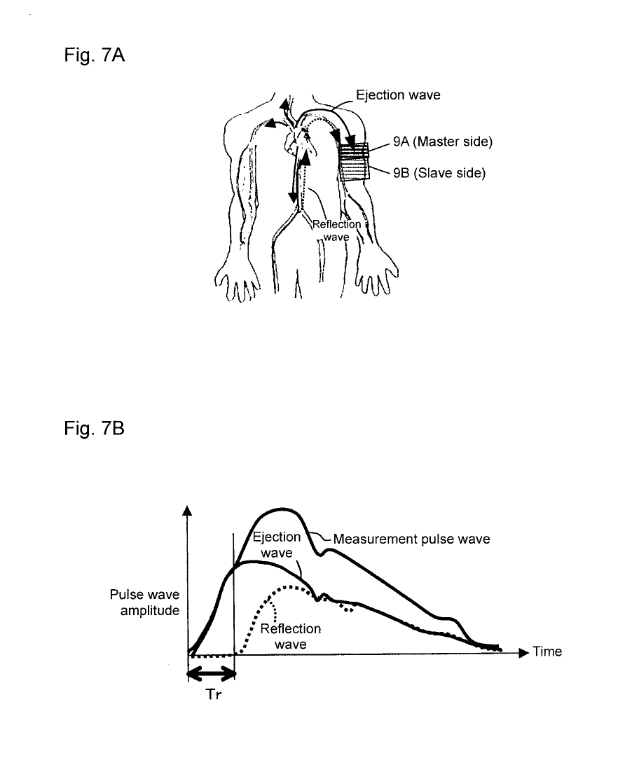

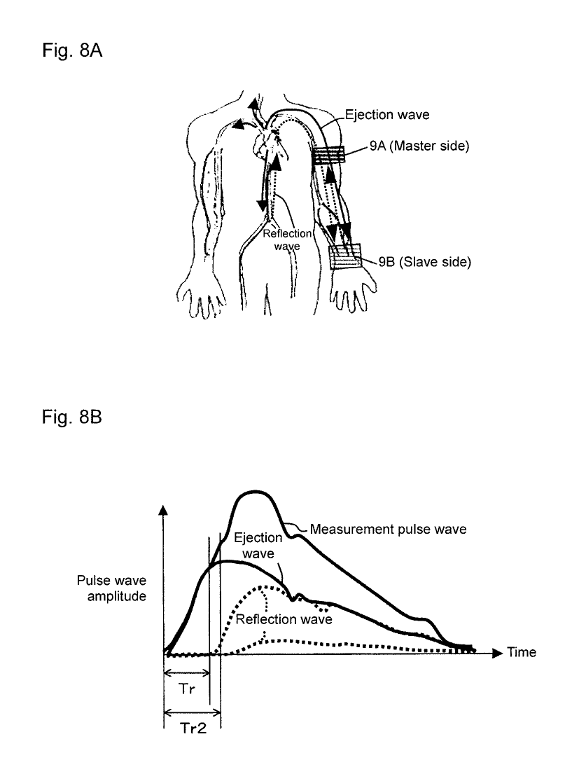

The method of calculating the index of arterial sclerosis in the measurement device 1A on the master side in step S39 will be described using FIG. 7A, FIG. 7B, FIG. 8A, and FIG. 8B.

In the first embodiment, the attachment site of the cuff 9B on the slave side may take two areas, the upper arm on the peripheral side than the attachment site of the cuff 9A on the master side shown in FIG. 7A or the wrist shown in FIG. 8A when the cuff 9A on the master side is attached to the upper arm. The peripheral side immediately from the measurement site on the master side in the example of FIG. 7A, and the wrist in the example of FIG. 8A are applied with the cuff 9B on the slave side.

FIG. 7B is a view describing the relationship of the pulse wave waveform measured when the attachment site of the cuff 9A on the master side and the attachment site of the cuff 9B on the slave side are in the relationship of FIG. 7A, the ejection wave, and the reflection wave. When the cuff is attached as in FIG. 7A, the waveform of when the ejection wave is reflected and returned from the branch portion of the iliac artery is detected as the reflection wave. The time difference Tr of the appearance of the reflection wave from the appearance of the ejection wave is obtained in the time from the rise of the measured pulse wave waveform to the first inflection point, as described using FIG. 3. In this case, the CPU 40 calculates the value obtained by dividing the trunk length proportional to the height by the time difference Tr as the PWV or the index of arterial sclerosis in step S39.

FIG. 8B is a view describing the relationship of the pulse wave waveform measured when the attachment site of the cuff 9A on the master side and the attachment site of the cuff 9B on the slave side are in the relationship of FIG. 8A, the ejection wave, and the reflection wave. When the cuff is attached as in FIG. 8A, the reflection wave includes the waveform reflected and returned from the attachment site of the cuff 9B on the slave side in addition to the waveform of when the ejection wave is reflected and returned from the branch portion of the iliac artery. The time differences Tr, Tr2 of the appearance of the respective waveform from the appearance of the ejection wave are obtained in the times from the rise of the measured pulse wave waveform to the first inflection point, and the next inflection point, as shown in FIG. 8B. In this case, the CPU 40 calculates the value obtained by dividing the trunk length proportional to the height by the time difference Tr as the first PWV, and calculates the value obtained by dividing the upper arm length proportional to the height by the time difference Tr2 as the second PWV in step S39.

The measurement device according to the first embodiment functions as the master and as the slave by accepting the selection from the operator. Thus, the cuff can be attached to plural areas and the attachment site can be compressed with the air bag by using a plurality of measurement devices as each function. Thus, the measurement device itself can be formed small compared to when compressing the attachment site with a plurality of air bags using one measurement device.

Furthermore, the measurement device according to the first embodiment performs the operation of compressing the blood vessel for avascularization without functioning as the pulse wave meter when functioning as the slave. Furthermore, it may be operated as a sphygmomanometer such as a wrist sphygmomanometer by functioning as a master when the slave does not exist, that is, by using the measurement device independently. Thus, the measurement device may be carried around as a wrist sphygmomanometer and the like when outside, and may be used in cooperation with another measurement device that functions as the master side or the slave side to measure the blood pressure information such as the index of the arterial sclerosis when at home.

### Second Embodiment

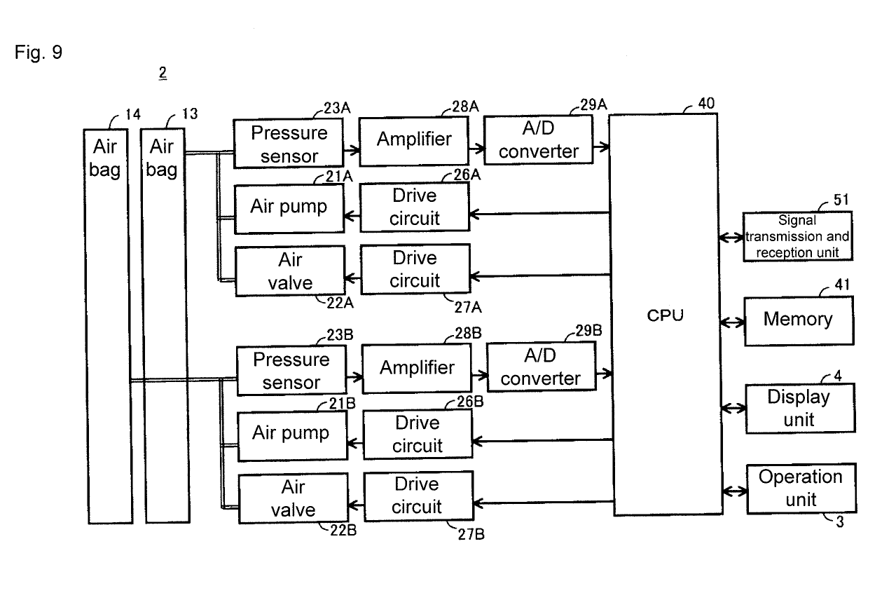

The function of a measurement device 2 according to a second embodiment will be described using FIG. 9. With reference to FIG. 9, the cuff 9 connected to the measurement device 2 includes an air bag 14 for pulse wave measurement in addition to the air bag 13 for blood pressure measurement. In addition to the configuration for controlling the air bag 13 of the measurement device 1, the measurement device 2 includes an air pump 21B, an air valve 22B, a pressure sensor 23B, drive circuits 26B, 27B, an amplifier 28B, and an A/D converter 29B for controlling the air bag 14. The function of each unit is similar to each corresponding unit of the measurement device 1.

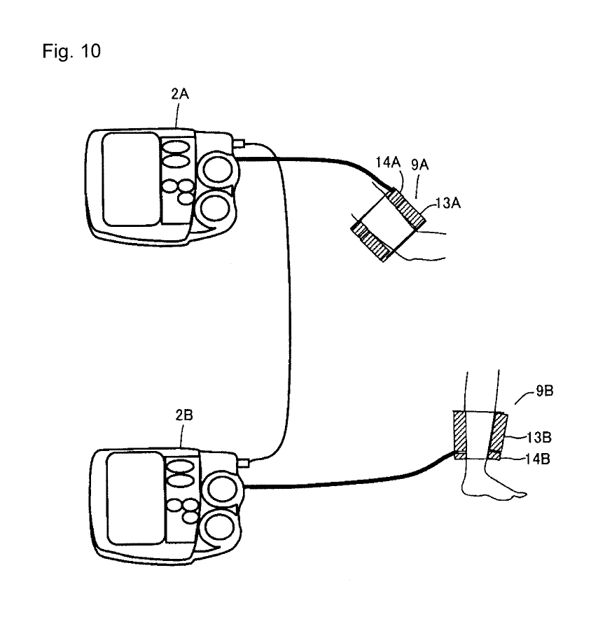

The measurement method using the measurement device 2 will now be described using FIG. 10. With reference to FIG. 10, two connected measurement devices 2 represented as the measurement devices 2A, 2B are used in the second embodiment, and are operated in cooperation with each other to obtain the blood pressure information and calculate the index of arterial sclerosis. In the case shown in FIG. 10, the measurement device 2A functions as a master, and the measurement device 2B functions as a slave. The measurement device 2A, which is a master, has the cuff 9A to be attached to the upper arm on the central side, and the measurement device 2B, which is the slave, has the cuff 9B to be attached to the ankle on the peripheral side.

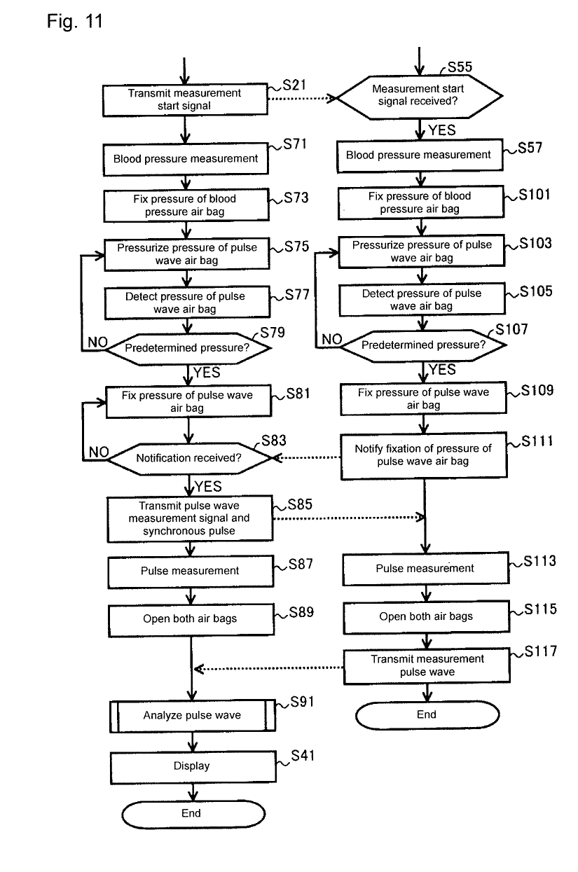

The measurement operation in the measurement device 2 will be described using FIG. 11. In the flowchart of FIG. 11, the measurement operation different from the measurement operation in the measurement device 1 shown in FIG. 6 of the measurement operation in the measurement device 2 is shown. The operation shown in the flowchart of FIG. 11 also starts when the switch 31 is pushed to turn ON the power arranged on the operation unit 3, and is realized when the CPU 40 reads out the program stored in the memory 41 and controls each unit shown in FIG. 9.

With reference to FIG. 11, in the measurement device 2A on the master side, when transmitting a signal instructing the start of the blood pressure measurement in step S21 to the measurement device 2B on the slave side, the CPU 40 outputs a control signal to the drive circuit 26A to measure the blood pressure while pressurizing the air bag 13A for blood pressure measurement in step S71. After the blood pressure measurement, the CPU 40 fixes the inner pressure of the air bag 13A to the pressure at the end of the measurement in step S73. The peripheral side is thereby avascularized by the air bag 13A on the peripheral side than the air bag 14A for pulse wave measurement. In step S75, the CPU 90 outputs the control signal to the drive circuit 26B and pressurizes the air bag 14A for pulse wave measurement. The CPU 90 pressurizes the air bag 14A until reaching a predetermined pressure while detecting the inner pressure of the air bag 14A based on the pressure signal from the pressure sensor 23B in step S77. When the inner pressure of the air bag 14A reaches the predetermined pressure (YES in step S79), the CPU 40 fixes the inner pressure of the air bag 14A at the predetermined pressure in step S81.

In the measurement device 2B on the slave side as well, when the signal instructing the start of measurement transmitted from the measurement device 1A on the master side is received by the signal transmission and reception unit 51 in step S21 (YES in step S55), the CPU 40 starts the blood pressure measurement operation in step S57. In steps S101 to S109, the operations similar to steps S73 to S81 in the measurement device 2A on the master side are performed. When the inner pressure of the air bag 14B is fixed at the predetermined pressure in step S109, the CPU 40 notifies the measurement device 2A on the master side that the inner pressure of the air bag 14B is fixed with the signal transmission and reception unit 51 in step S111.

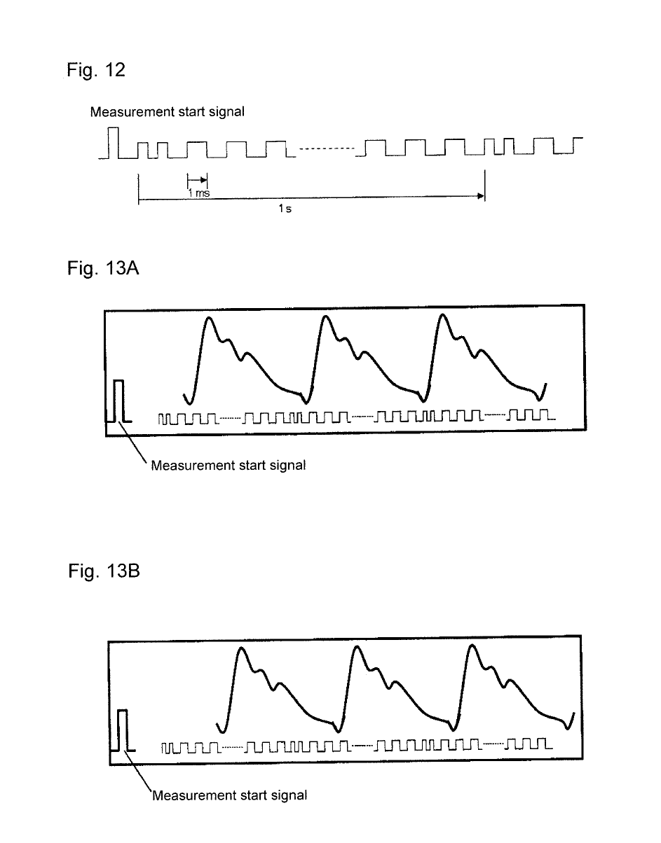

When the measurement device 2A on the master side receives the notification (YES in step S83), the CPU 40 transmits a signal instructing the start of measurement of the pulse wave to the measurement device 2B on the slave side with the signal transmission and reception unit 51 in step S85. The transmission of the synchronous pulse also starts. FIG. 12 is a view showing a specific example of the measurement start signal and the synchronous pulse transmitted in step S85. In the example shown in FIG. 12, the measurement start signal is added to the synchronous pulse having a width of a millisecond unit. Thus, the measurement device 2B on the slave side can synchronize with the operation of the measurement device 2A on the master side in a millisecond unit. The width of each time point of the synchronous pulse is preferably rendered a different width by the method defined in advance. Therefore, both the measurement device 2A on the master side and the measurement device 2B on the slave side can distinguish which time point the current time point is in one second.

In the measurement device 2A on the master side, the CPU 40 measures the pulse wave according to the timing indicated by the measurement start signal transmitted to the measurement device 2B on the slave side in step S87. The pulse wave is then stored as the measurement result with the measurement start signal and the synchronous pulse as shown in FIG. 13A. Similarly, in the measurement device 2B on the slave side, the CPU 40 measures the pulse wave according to the timing indicated by the measurement start signal transmitted from the measurement device 2A on the master side in step S113, and stores the pulse wave along with the measurement start signal and the synchronous pulse, as shown in FIG. 13B.

After the measurement of the pulse wave is terminated, the air bags 13A, 13B, 14A, 14B are opened in the measurement devices 2A, 2B, respectively, in steps S89, S115. In the measurement device 2B on the slave side, the CPU 40 transmits the measurement result of the pulse wave obtained in step S113 to the measurement device 2A on the master side with the signal transmission and reception unit 51 in step S117, and terminates the process.

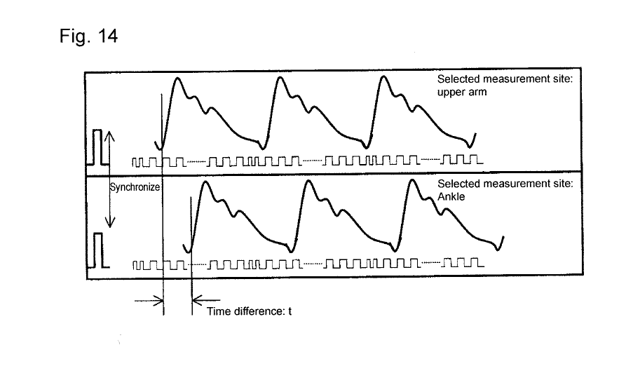

In the measurement device 2A on the master side, the CPU 40 analyzes the measurement result of the pulse wave obtained in step S87 and the measurement result of the pulse wave transmitted from the measurement device 2B on the slave side and obtains the index of arterial sclerosis in step S91. With reference to FIG. 14, the CPU 40 calculates the appearance time difference t of the pulse waves by synchronizing the pulse wave waveforms measured in the devices 2A, 2B shown in FIGS. 13A, 13B based on the measurement start signal in step S91. The CPU 40 then obtains baPWV (brachial-ankle PWV) by dividing the distance between the measurement site (upper arm) in the measurement device 2A and the measurement site (ankle) in the measurement device 2B with the calculated time difference t. The distance between the measurement sites may be defined in advance, or may be measured and input by the measurer, or a mechanism for measuring the distance therebetween may be arranged in the cuffs 9A, 9B and the distance may be input by such mechanism.

In step S91, the ratio of the blood pressure value measured at the ankle in step S57 with respect to the blood pressure value measured at the upper arm in step S71, or ABI (Ankle Brachial Pressure Index) may be calculated as the index of arterial sclerosis. The ABI is also a useful index for determining the degree of arterial sclerosis. The degree of arterial sclerosis is determined as normal if the ABI is greater than or equal to 1.0, and the arterial sclerosis is determined as advancing (e.g., possibility of arteriosclerotic obliteration) if the ABI is lower than or equal to 0.9.

In the measurement device 2A on the master side, the CPU 40 performs the process for displaying the calculated index on the display unit 4 along with the measured blood pressure, and the like for display, and terminates the series of processes.

The measurement device according to the second embodiment functions as the master and as the slave by accepting the selection from the operator. When functioning as the master, the pulse signal and the measurement start signal can be transmitted to the measurement device on the slave side, and the measurement timing on the slave side can be controlled. The timing to measure the pulse wave at plural areas thus can be controlled, and the appearance time difference t of the pulse wave can be easily obtained at high accuracy. Thus, the index of arterial sclerosis can be easily obtained with high accuracy.

### [Variant of Second Embodiment]

In the above example, one of the upper arms and one of the ankles are used for the plurality of measurement sites, as shown in FIG. 10, and the PWV or the index of arterial sclerosis is calculated based on the pulse wave obtained at each measurement site. The plurality of measurement sites is not limited to two areas as described above, and may be three or more areas. As a variant, the configuration of the measurement device when obtaining the index of arterial sclerosis at three measurement sites will be described.

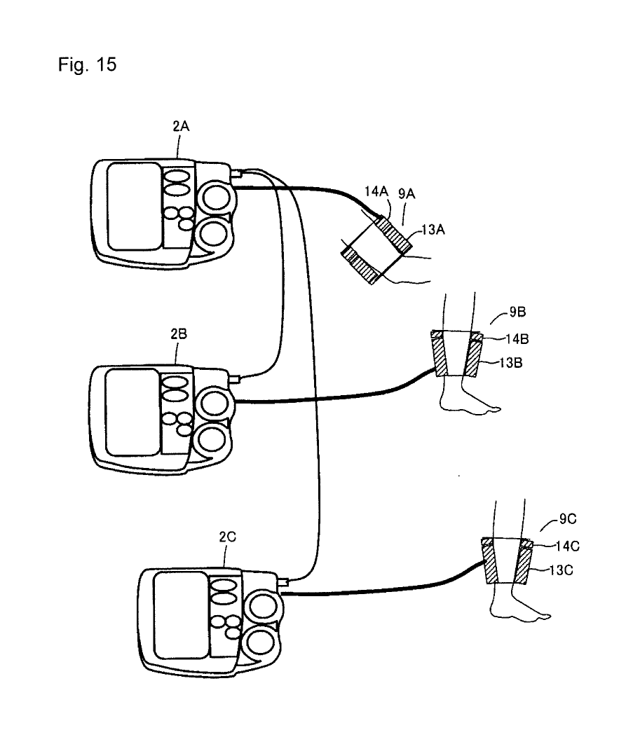

The measurement method using the measurement device 2 according to a variant of the second embodiment will be described using FIG. 15. With reference to FIG. 15, one measurement device functioning as a master and two measurement devices functioning as a slave connected to the relevant measurement device, which are represented as the measurement devices 2A, 2B, 2C, are used in the variant of the second embodiment, and are operated in cooperation with each other to obtain the blood pressure information and calculate the index of arterial sclerosis. In the case shown in FIG. 15, the measurement device 2A functions as a master, and the measurement devices 2B, 2C both function as a slave. The measurement device 2A, which is a master, has the cuff 9A to be connected attached to the upper arm on the central side, and the measurement devices 2B, 2C, which are the slaves, have the cuffs 9B, 9C to be connected attached to both ankles or the peripheral side.

In the case of the variant of the second embodiment, the measurement devices 2B, 2C on the slave side both perform the operation similar to the operation of the measurement device on the slave side shown in FIG. 11. The measurement device 2A on the master side checks the existence of the measurement devices 2B, 2C on the slave side in steps S17, S19, and checks whether or not the respective measurement site is appropriate. In step S87, the CPU 40 of the measurement device 2A on the master side compares the pulse wave waveform measured in the measurement device 2A on the master side and the pulse wave waveform measured in the measurement device 2B on the slave side, and the pulse wave waveform measured in the measurement device 2A on the master side and the pulse wave waveform measured in the measurement device 2C on the slave side as shown in FIGS. 13A and 13B, and obtains the degree of arterial sclerosis in each comparison.

With such configuration, the index of arterial sclerosis is obtained based on the pulse wave waveforms at a plurality of measurement sites, and the accuracy of the index of arterial sclerosis can be enhanced.

### [First Variant]

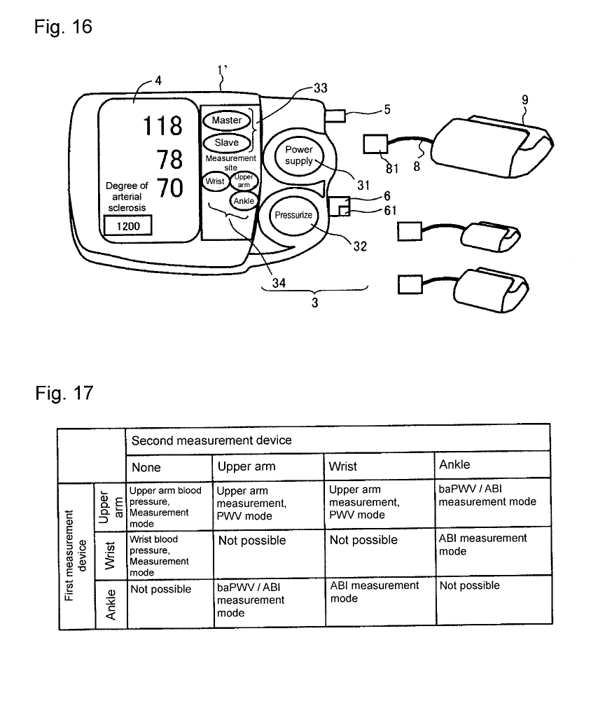

The measurement device 1 and the measurement device 2 select the measurement site based on the operation signal from the switch 34. The measurement device 1′ according to the first variant, on the other hand, is configured as shown in FIG. 16. With reference to FIG. 16, the cuff 9 is provided for every site to be attached in the first variant. The air tube 8 for connecting the cuff 9 includes a storage unit 81 for storing the determination information showing the site where the cuff 9 is to be attached. The measurement device 1′ includes an air connector 6 for connecting the air tube 8, and the air connector 6 includes a readout unit 61 for connecting to the storage unit 81 and reading out the determination information by connecting the air tube 8. The specific configuration of the storage unit 81 and the readout unit 61 may be a storage device such as an IC chip and a device for reading out the information from the relevant device. Such electrical configuration is not the only case, and a mechanical configuration may be adopted. In other words, the storage units 81 may have different shapes, for example, having pins of different shapes, for every site where the cuff 9 is to be attached, and the readout unit 61 may include a button or may include a light emitting element/light receiving element and read out the difference in shape. The information read by the readout unit 61 is input to the CPU 40. The CPU 40 thereby determines the measurement site.

With such configuration, the measurement site is automatically determined by attaching the cuff 9 to the measurement site without the operation for selecting the measurement site by the measurer, and the blood pressure information can be obtained.

### [Second Variant]

The measurement device 1 calculates the index of arterial sclerosis by avascularizing the wrist or the lower side of the upper arm and measuring the pulse wave at the upper arm. The measurement device 2 calculates the index of arterial sclerosis by measuring the pulse wave at both the upper arm and the ankle. In such devices, the pulse wave is not measured as an error if other positions are set as the measurement site. In the second variant, on the other hand, the measurement operation according to the first embodiment and the measurement operation according to the second embodiment may be carried out in combination in the measurement device. Further, whether the operation mode corresponding to the combination of the measurement sites is the operation mode in which the operation described in the first embodiment is carried out or the operation mode in which the operation described in the second embodiment is carried out may be automatically determined.

Specifically, the measurement device according to the second variant stores the operation mode for every combination of measurement sites, as shown in FIG. 17 in the memory 41. FIG. 17 shows a specific example of the relationship of the combination of measurement sites when measurement is performed using two measurement devices represented as the first measurement device and the second measurement device, and the operation mode in the first measurement device.

With reference to FIG. 17, if the cuff of the first measurement device is attached to the upper arm and the cuff of the second measurement device is not attached, the first measurement device is used alone and the blood pressure is measured with the upper arm as the measurement site, as described in the first embodiment. If the cuff of the second measurement device is attached to the upper arm or the wrist, the PWV serving as the index of arterial sclerosis based on the pulse wave measured at the upper arm is calculated in the first measurement device, as described in the first embodiment. If the cuff of the second measurement device is attached to the ankle, the baPWV serving as the index of arterial sclerosis based on the pulse wave measured at the upper arm and the ankle is calculated in the first measurement device, as described in the second embodiment. Alternatively, the ABI serving as the index of arterial sclerosis based on the blood pressure measured at the upper arm and the ankle is calculated.

If the cuff of the first measurement device is attached to the wrist and the cuff of the second measurement device is not attached, the first measurement device is used alone and the blood pressure is measured with the wrist as the measurement site, similar to the operation described in the first embodiment. If the cuff of the second measurement device is attached to the upper arm or the wrist, the operation is not carried out and the first measurement device does not function as the master. If the cuff of the second measurement device is attached to the ankle, the ABI serving as the index of arterial sclerosis based on the blood pressure measured at the wrist and the ankle is calculated in the first measurement device, similar to the operation described in the second embodiment.

If the cuff of the first measurement device is attached to the wrist and the cuff of the second measurement device is not attached or is attached to the ankle, the operation is not carried out and the first measurement device does not function as the master. If the cuff of the second measurement device is attached to the upper arm, the baPWV serving as the index of arterial sclerosis based on the pulse wave measured at the upper arm and the ankle is calculated in the first measurement device, similar to the operation described in the second embodiment. Alternatively, the ABI serving as the index of arterial sclerosis based on the blood pressure measured at the upper arm and the ankle is calculated. If the cuff of the second measurement device is attached to the wrist, the ABI serving as the index of arterial sclerosis based on the blood pressure measured at the wrist and the ankle is calculated in the first measurement device, similar to the operation described in the second embodiment.

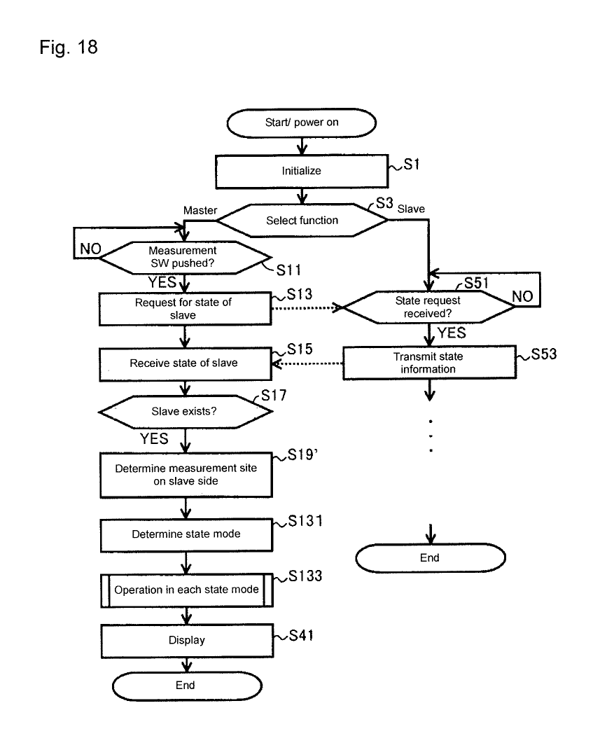

The measurement operation in the measurement device according to the second variant will be described using FIG. 18. In the flowchart of FIG. 18, the measurement operation different from the measurement operation in the measurement device 1 shown in FIG. 6 of the measurement operation according to the second variant is shown.

With reference to FIG. 18, the CPU 40 determines where the measurement site on the slave side is in step S19′ after the measurement device on the slave side is confirmed to exist (YES in step S17) in the measurement device on the master side in the second variant. In step S131, the CPU 40 determines the corresponding measurement mode based on the relationship shown in FIG. 17 from the measurement site of the relevant measurement device and the measurement site of the measurement device on the slave side. In step S133, the measurement operation is carried out in the measurement mode determined in step S131 as described in the first embodiment or the second embodiment.

The first variant may be combined with the second variant, the measurement site may be detected in each measurement site, and the operation mode may be determined based on the measurement site detected in each measurement device in the measurement device on the master side.

With such configuration, the appropriate operation mode is determined by attaching the cuff 9 to the measurement site without the operation for selecting the operation mode by the measurer, and the blood pressure information can be obtained.

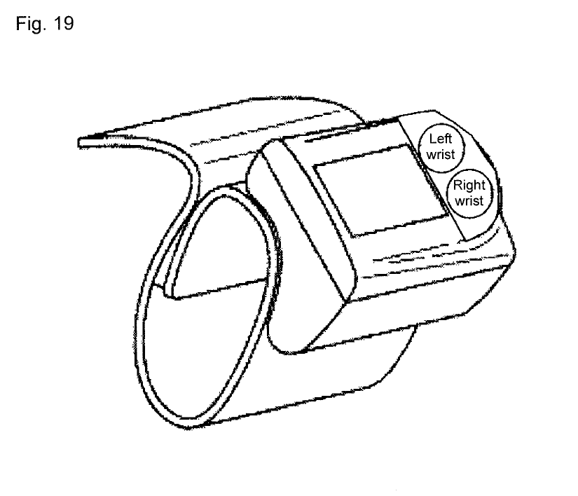

The above examples all show a configuration of obtaining the blood pressure information by compressing a plurality of areas with the air bag using a plurality of the same measurement devices. In other words, the measurement devices 1, 2 according to the embodiment store the program for functioning as the master and the program for functioning as the slave in the memory 41, and operate by reading out the corresponding program in accordance with the selection. However, the program for causing the measurement device to function as the master may be stored without storing the program for causing the measurement device to function as the slave so as to function as the measurement device alone and only as the master. Alternatively, the program for functioning as the slave may be stored without storing the program for functioning as the master so as to function as the measurement device alone and only as the master. Furthermore, with regards to the measurement device functioning as the slave, the measurement site can be limited to the ankle or the wrist, in which case, the sphygmomanometer for ankle or wrist can be used as shown in FIG. 19.

The embodiments disclosed herein are illustrative in all aspects and should not be construed as being restrictive. The scope of the invention is defined by the claims rather than by the description made above, and meanings equivalent to the claims and all modifications within the scope are intended to be encompassed therein.

### DESCRIPTION OF SYMBOLS

1, 1A, 1B, 1′, 2, 2A, 2B, 2C measurement device
3 operation unit
4 display unit
5 connector
6 air connector
8 air tube
9, 9A, 9B, 9C cuff
13, 13A, 13B, 14, 14A, 14B air bag
21, 21A, 21B air pump
22, 22A, 22B air valve
23, 23A, 23B pressure sensor
26, 26A, 26B, 27, 27A, 27B drive circuit
28, 28A, 28B amplifier
29, 29A, 29B A/D converter
31, 32, 33, 34 switch
40 CPU
41 memory
51 signal transmission and reception unit
61 readout unit
81 storage unit

1. A blood pressure information measurement device comprising:
a fluid bag;
a measurement unit, connected to the fluid bag for acquiring blood pressure information based on a pressure change of the fluid bag; and
a communication unit for communicating with another blood pressure information measurement device,
wherein the communication unit transmits a signal for instructing a start of measurement to the other blood pressure information measurement device and acquires the blood pressure information measured by the other blood pressure information measurement device from the other blood pressure measurement device, and
wherein the blood pressure information measurement device further includes a calculation unit for calculating an index of arterial sclerosis based on first blood pressure information, which is the blood pressure information measured by the measurement unit, and second blood pressure information, which is the blood pressure information measured by the other blood pressure information measurement device.

2. The blood pressure information measurement device according to claim 1,
wherein the blood pressure information is a pulse wave waveform, and
wherein the calculation unit synchronizes the pulse wave waveform for the first blood pressure information and the pulse wave waveform for the second blood pressure information based on the signal for instructing the start of measurement to detect a time difference between times at which rising points of the pulse wave waveforms appear, and calculates a pulse wave velocity using the time difference as the index of arterial sclerosis.

3. The blood pressure information measurement device according to claim 2,
wherein the communication unit transmits a synchronization pulse in addition to the signal for instructing the start of measurement, acquires the pulse wave waveform corresponding to the synchronization pulse from the other blood pressure information measurement device, and
wherein the calculation unit synchronizes the pulse wave waveform for the first blood pressure information and the pulse wave waveform for the second blood pressure information using the synchronization pulse corresponding to the pulse wave waveform.

4. The blood pressure information measurement device according to claim 1, further comprising: a selection unit for accepting a selection of a measurement site at the measurement unit,
wherein the communication unit acquires information for specifying the measurement site in the other blood pressure measurement device.

5. The blood pressure information measurement device according to claim 4,
wherein the fluid bag corresponds to the measurement site, and
wherein a determination unit for determining a corresponding measurement site from the fluid bag connected to the measurement unit is arranged in place of the selection unit.

6. The blood pressure information measurement device according to claim 1,
wherein the blood pressure information is a blood pressure value, and
wherein the calculation unit calculates a ratio between a blood pressure value for the first blood pressure information and a blood pressure value for the second blood pressure information as the index of arterial sclerosis.

7. The blood pressure information measurement device according to claim 1, having a first processing function and a second processing function, further comprising:
a selection unit for accepting a selection of the first processing function or the second processing function for a processing function,
wherein the communication unit transmits the blood pressure information measured in the measurement unit,
wherein the communication unit transmits the signal for instructing the start of measurement of the blood pressure information to the other blood pressure information measurement device if the first processing function is selected in the selection unit,
wherein the measurement unit measures the blood pressure information based on the signal for instructing the start of measurement of the blood pressure information transmitted from the other blood pressure information measurement device and the communication unit transmits the measurement result to the other blood pressure information measurement device if the second processing function is selected in the selection unit, and
wherein the calculation unit calculates the index of arterial sclerosis using first blood pressure information or the blood pressure information measured by the measurement unit and second blood pressure information or the blood pressure information measured by the other blood pressure information measurement device received by the communication unit if the first processing function is selected in the selection unit.

8. A blood pressure information measurement device comprising:
a fluid bag;
a measurement unit for measuring a pulse wave based on a pressure change of the fluid bag; and
a communication unit for communicating with another blood pressure information measurement device,
wherein the communication unit transmits a control signal for controlling an inner pressure of the fluid bag to the other blood pressure information measurement device, and
wherein the blood pressure information measurement device further includes a calculation unit for calculating an index of arterial sclerosis from the pulse wave measured by the measurement unit while controlling the inner pressure of the fluid bag of the other blood pressure information measurement device with the control signal.

9. The blood pressure information measurement device according to claim 8, further comprising: a selection unit for accepting a selection of a measurement site of the fluid bag,
wherein the communication unit acquires information for specifying an attachment site of the fluid bag in the other blood pressure measurement device, and the calculation unit calculates the index of arterial sclerosis using a distance between an attachment site of the fluid bag and the attachment site of the fluid bag in the other blood pressure measurement device.

10. The blood pressure information measurement device according to claim 9,
wherein the fluid bag corresponds to the attachment site, and a determination unit for determining a corresponding attachment site from the fluid bag connected to the measurement unit is arranged in place of the selection unit.

11. The blood pressure information measurement device according to claim 9,
wherein the calculation unit includes a mechanism for calculating the distance between the attachment site of the fluid bag and the attachment site of the fluid bag in the other blood pressure measurement device.

12. A blood pressure information measurement system comprising:
a first blood pressure information measurement device; and
a second blood pressure information measurement device,
wherein the first blood pressure information measurement device and the second blood pressure information measurement device acquire blood pressure information at different measurement sites of a same living body, and
wherein an index of arterial sclerosis of the living body is calculated based on the blood pressure information measured in the blood pressure information measurement devices in at least one blood pressure information measurement device of the first blood pressure information measurement device or the second blood pressure information measurement device.
